(19) United States
(12) Patent Application Publication
(10) Pub. No.: US 2020/0410577 A1
(43) Pub. Date: Dec. 31, 2020

(54) VEHICLE LENDING CONTROL DEVICE AND VEHICLE LENDING CONTROL METHOD FOR MANAGING A PLURALITY OF VEHICLES AVAILABLE FOR RENT

(71) Applicant: Panasonic Intellectual Property Management Co., Ltd., Osaka (JP)

(72) Inventors: Akira MASUO, Kanagawa (JP); Junichi YUKAWA, Kanagawa (JP); Kenji SUGIHARA, Tokyo (JP)

(73) Assignee: Panasonic Intellectual Property Management Co., Ltd., Osaka (JP)

(21) Appl. No.: 17/015,890

(22) Filed: Sep. 9, 2020

Related U.S. Application Data
(63) Continuation of application No. PCT/JP2019/009976, filed on Mar. 12, 2019.

(30) Foreign Application Priority Data
Mar. 16, 2018 (JP) 2018-050148

(51) Int. Cl. G06Q 30/06; G06Q 50/30; G06Q 10/06; G16H 40/67; A61B 5/00; A61B 5/18; A61B 5/11; A61B 5/16

(52) U.S. Cl. CPC G06Q 30/0645; G06Q 50/30; G06Q 10/06315; G16H 40/67; A61B 5/4812; A61B 5/4815; G06Q 2240/00; A61B 5/1123; A61B 5/165; A61B 5/0077; A61B 5/4803; A61B 5/1114; A61B 5/0002; A61B 5/18

(57) ABSTRACT

When the communication circuit receives, from the second electronic device, information indicating application to rent a vehicle by a given user and biological information on the given user, and when the biological information on the given user meets a first condition, the communication circuit outputs information to offer to lend a vehicle of the first rank. When the communication circuit receives, from the second electronic device, information indicating application to rent a vehicle by a given user and biological information on the given user, and when the biological information on the given user meets a second condition different from the first condition, the communication circuit outputs information to offer to lend a vehicle of the second rank.

### BACKGROUND

### 1. Field

The present disclosure relates to a vehicle lending control device and a vehicle lending control method for managing a plurality of vehicles available for rent.

### 2. Description of the Related Art

Car sharing systems to let a plurality of pre-registered members share a vehicle and car rental systems to rent a vehicle from a business operator by paying a predetermined charge have been put into practical use. In these systems, a determination is made by using behavior information related to the behavior of a user using a vehicle in order to impose a restriction on the use of a vehicle by a user with bad behavior and to let a good user use a vehicle without restriction. Behavior includes driving skill, driving attitude, manners, etc. of a user (see, for example, patent document 1).
[Patent Literature 1] JP2009-271632

By using behavior information, it is possible to discriminate between users with bad behavior and good users. However, it is not possible to impose a restriction on the use of a vehicle by a first-time user for which behavior information is not available.

### SUMMARY

The disclosure addresses the above-described issue, and a general purpose thereof is to provide a technology for managing a plurality of vehicles available for rent such that a restriction is imposed on the use of a vehicle by a user regardless of whether the user has used a vehicle in the past.

The vehicle lending control device according to an embodiment of the present disclosure is a vehicle lending control device including at least a communication circuit adapted to communicate with an electronic device, wherein a first rank related to a vehicle and a second rank related to a vehicle and lower than the first rank are provided, when the communication circuit receives, from the electronic device, information indicating application to rent a vehicle by a given user and biological information on the given user, and when the biological information on the given user meets a first condition, the communication circuit outputs information to offer to lend a vehicle of the first rank, and, when the communication circuit receives, from the electronic device, information indicating application to rent a vehicle by a given user and biological information on the given user, and when the biological information on the given user meets a second condition different from the first condition, the communication circuit outputs information to offer to lend a vehicle of the second rank.

Another embodiment of the present disclosure relates to a vehicle lending control method. The method is a vehicle lending control method that can be used in a vehicle lending control device including at least a communication circuit adapted to communicate with an electronic device, wherein a first rank related to a vehicle and a second rank related to a vehicle and lower than the first rank are provided, when the communication circuit receives, from the electronic device, information indicating application to rent a vehicle by a given user and biological information on the given user, and when the biological information on the given user meets a first condition, the communication circuit outputs information to offer to lend a vehicle of the first rank, and, when the communication circuit receives, from the electronic device, information indicating application to rent a vehicle by a given user and biological information on the given user, and when the biological information on the given user meets a second condition different from the first condition, the communication circuit outputs information to offer to lend a vehicle of the second rank.

Optional combinations of the aforementioned constituting elements, and implementations of the present disclosure in the form of devices, systems, methods, programs, recording mediums having programs recorded thereon, and vehicles carrying the device may also be practiced as additional modes of the present disclosure.

### BRIEF DESCRIPTION OF THE DRAWINGS

The figures depict one or more implementations in accordance with the present teaching, by way of example only, not by way of limitations. In the figures, like reference numerals refer to the same or similar elements.

FIG. 1 shows a configuration of a vehicle lending control system according to the embodiment;

FIG. 2 shows a data structure of a table stored in the storage circuit of FIG. 1;

FIG. 3 shows a configuration of the second electronic device of FIG. 1;

FIGS. 4A-4E show screens displayed on the electronic device of FIG. 1;

FIGS. 5A-5B show data structures of tables stored in the determination circuit of FIG. 1;

FIGS. 6A-6C show data structures of alternative table stored in the determination circuit of FIG. 1;

FIG. 7 is a flowchart showing a sequence of steps for determination by the vehicle lending control device of FIG. 1; and

FIG. 8 shows a data structure of a table stored in the determination circuit according to a variation.

### DETAILED DESCRIPTION

The invention will now be described by reference to the preferred embodiments. This does not intend to limit the scope of the present invention, but to exemplify the invention.

A brief summary will be given before describing the present disclosure in specific details. An embodiment relates to a vehicle lending control system for managing a plurality of vehicles available for rent in a car sharing system or a rental car system. The vehicle lending control system defines a plurality of ranks and groups a plurality of vehicles into the plurality of ranks such that a vehicle is included in at least one rank. For example, three ranks are defined. The highest rank (hereinafter, “rank 1”) includes all vehicles, the intermediate rank (hereinafter “rank 3”) includes automatically driven vehicles and support cars, and the lowest rank (hereinafter “rank 5”) does not include vehicles. Further, the vehicle lending control system also groups uses wishing to rent a vehicle into three ranks and determines to lend a vehicle included in the rank in which the user is grouped. In the past, ranking of users has been performed based on behavior information related to the behavior of a user using a vehicle. Specifically, a rank has been determined according to whether the vehicle is filled up, whether the vehicle is clean inside, whether the driving attitude has been good, etc. However, it is impossible to determine a rank of a first-time user for which behavior information is not available. It is required to determine a rank of the user even in such a situation.

The vehicle lending control system according to the embodiment includes a vehicle lending control device and an electronic device. The electronic device is exemplified by a watch and a smartphone and is connected to a vital sensor. The vital sensor acquires biological information on the driver such as sleep efficiency and sleeping time. The electronic device transmits the biological information to the vehicle lending control device. The vehicle lending control device associates the user (person wising to rent a vehicle) with one of a plurality of ranks based on the received biological information. The vehicle lending control device also determines that a vehicle of the rank associated with the user should be rented. The behavior information mentioned above indicates dangerousness of driving directly or indirectly. Meanwhile, dangerousness of driving can also be determined by referring to the sleepiness of the driver. When the driver is in a sleepy condition, for example, the dangerousness of driving is increased. In this embodiment, therefore, a user is grouped into a rank based on biological information indicating the sleepiness of the driver instead of behavior information. A description will now be given of the embodiment with reference to the drawings. The embodiment described below is by way of one example only, and the present disclosure is not limited to the embodiment.

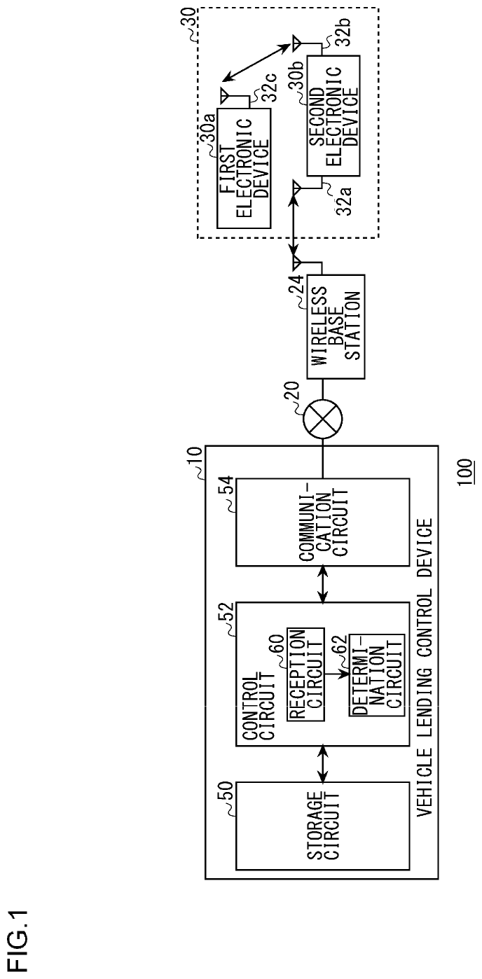

FIG. 1 shows a configuration of a vehicle lending control system 100. The vehicle lending control system 100 includes a vehicle lending control device 10, a network 20, a wireless base station 24, and an electronic device 30. The vehicle lending control device 10 includes a storage circuit 50, a control circuit 52, and a communication circuit 54. The control circuit 52 includes a reception circuit 60 and a determination circuit 62. The electronic device 30 includes a first electronic device 30a and a second electronic device 30b. The first electronic device 30a includes a third antenna 32c. The second electronic device 30b includes a first antenna 32a and a second antenna 32b.

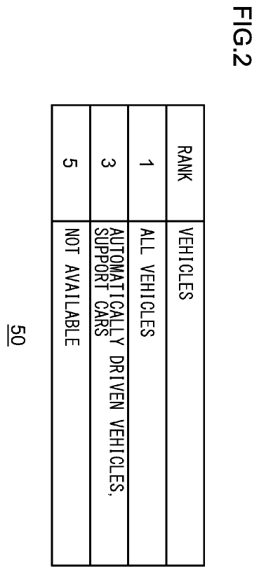

The storage circuit 50 of the vehicle lending control device 10 defines a plurality of ranks to which a plurality of vehicles should be grouped. As mentioned above, three ranks are defined and are referred to as rank 1, rank 3, and rank 5 in the descending order of ranking. In other words, rank 3 is lower than rank 1, and rank 5 is lower than rank 3. Alternatively, rank 1, rank 2, and rank 3 may be defined. Further, the storage circuit 50 groups and manages the plurality of vehicles available for rent such that each vehicle is included in at least one of the plurality of ranks. FIG. 2 shows a data structure of a table stored in the storage circuit 50. The table lists types of vehicles included in each rank. Rank 1 includes all vehicles, rank 3 includes automatically driven vehicles and support cars, and rank 5 does not include vehicles. Being ranked as Rank 5 means that the vehicle cannot be rented. In other words, the higher than rank, the larger the number of vehicles included in the rank. The table may include identification on vehicles included in the respective ranks, but a description thereof is omitted. Grouping of vehicles into respective ranks is not limited to this. The plurality of ranks may be defined based on Maslow's five stages, but a description thereof is omitted. Reference is made back to FIG. 1.

The first electronic device 30a is exemplified by a watch and a smartphone. The second electronic device 30b is a sensor capable of acquiring biological information on the user and is exemplified by a vital sensor and a driver monitoring device mounted in the vehicle. The second electronic device 30b measures biological information by using the vital sensor. The biological information is one of (1) information on sleeping time indicating actual sleeping time, (2) information on sleep efficiency indicating actual depth of sleep (REM/non-REM), (3) information indicating a proportion of a period of time in which the driver opens and closes the eyes during driving, (4) information indicating a proportion of a period of time in which the driver yawns (opens the mouth wider than a certain width), (5) sleepiness information acquired from the amount of heat dissipated from the body during driving, (6) emotion information acquired from a facial expression during driving and indicating a proportion of a period of time in which the driver is feeling bad (angry/sad), (7) emotion information acquired from the voice during driving and indicating a proportion of a period of time in which the driver is feeling bad (angry/sad), and (8) physical condition information acquired from the tilt of the head during driving and indicating a proportion of a period of time in which the physical condition becomes poorer. Items measured are not limited to these, and items that make it possible to understand the degree of sleepiness of the driver may be measured. The biological information may be information measured before the driver makes a request to rent the vehicle. Further, the first electronic device 30a includes a wireless communication function such as that of Bluetooth (registered trademark) and transmits the biological information from the third antenna 32c to the second electronic device 30b.

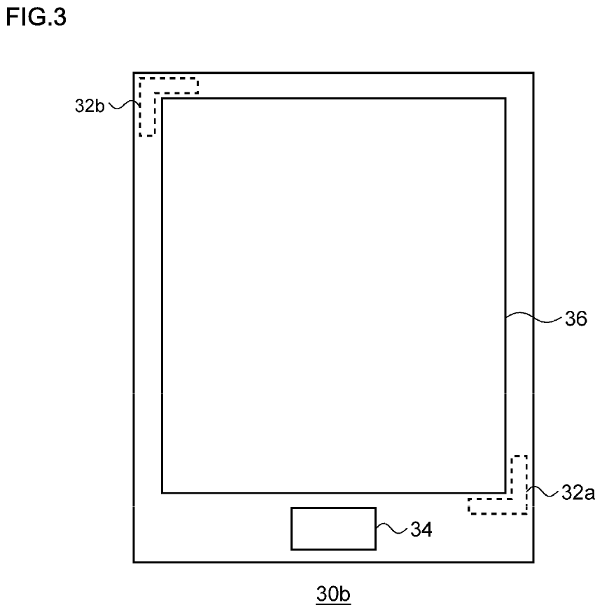

The second electronic device 30b is exemplified by a smartphone. FIG. 3 shows a configuration of the second electronic device 30b. The second electronic device 30b includes a first antenna 32a, a second antenna 32b, a home key 34, and a touch panel 36. The home key 34 can be pressed down by the user. The second antenna 32b can communicate with the first electronic device 30a and receives biological information from the first electronic device 30a. The touch panel 36 can display information and is also a user operation interface adapted to receive an application to rent a vehicle from the user. When the touch panel 36 receives an application to rent a vehicle, the second electronic device 30b starts an application program for lending.

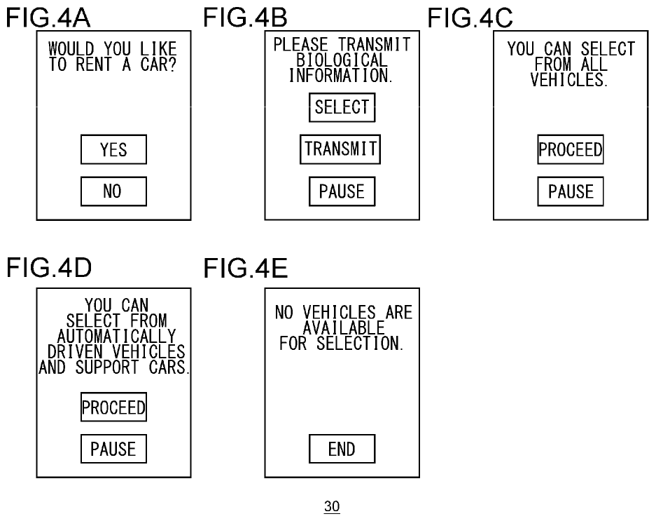

FIGS. 4A-4E show screens displayed on the second electronic device 30b. FIG. 4A is a screen shown after the program is started and shows a “Yes” button and a “No” button along with a message “Would you like to rent a car?”. When the user presses the “No” button, the process is terminated. When the user presses the “Yes” button, the second electronic device 30b transmits information indicating application to rent a vehicle by a given user (hereinafter, “request signal”) from the first antenna 32a to the vehicle lending control device 10. The request signal includes identification information on the second electronic device 30b or identification information on the user using the second electronic device 30b (hereinafter, generically referred to as “identification information”). The first antenna 32a and the wireless base station 24 are connected by wireless communication like 3G, 4G, 5G cellular or wireless local area network (LAN).

The communication circuit 54 of the vehicle lending control device 10 communicates with the second electronic device 30b via the network 20. When the request signal from the electronic device 30 is received, the communication circuit 54 outputs the request signal to the control circuit 52. When the request signal is received, the control circuit 52 acquires the identification information and starts a lending process for the user. The control circuit 52 generates a signal to respond to the request signal (hereinafter, “response signal”) and outputs the response signal to the communication circuit 54. The communication circuit 54 transmits the response signal to the second electronic device 30b via the network 20.

When the response signal is received, the second electronic device 30b displays a screen shown in FIG. 4B. A “Select” button, a “Transmit” button, and a “Pause” button are shown along with a message “Please transmit biological information”. When the user selects the “Select” button, the biological information already measured is made selectable. When the user selects the “Transmit” button, the second electronic device 30b transmits a signal including the selected biological information (hereinafter, “information signal”) to the vehicle lending control device 10. When the user selects the “Pause” button, the process is terminated. In other words, the second electronic device 30b transmits the biological information on the user to the vehicle lending control device 10 after the request signal. As a result, the biological information acquired by the first electronic device 30a enters the vehicle lending control device 10 via the second electronic device 30b.

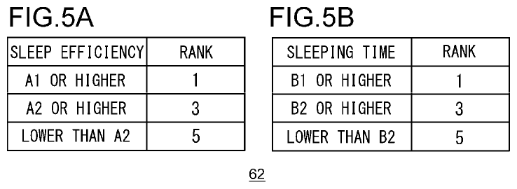

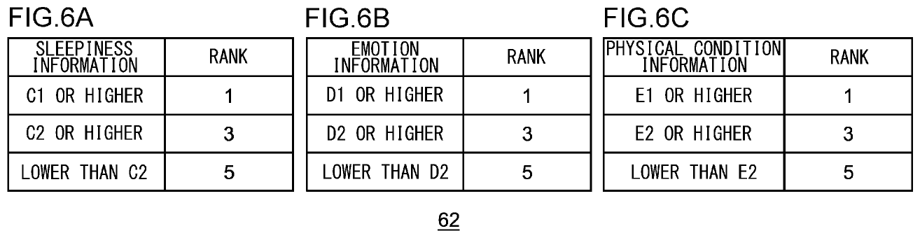

When the information signal from the second electronic device 30b is received, the communication circuit 54 of the vehicle lending control device 10 outputs the information signal to the control circuit 52. In other words, the communication circuit 54 receives the request signal before the information signal. The reception circuit 60 receives the information signal from the communication circuit 54. The determination circuit 62 determines a rank based on the biological information. FIGS. 5A-5B show data structures of tables stored in the determination circuit 62. FIG. 5A shows a table used when the biological information includes sleep efficiency. The determination circuit 62 determines that the user is rank 1 when the sleep efficiency is equal to or higher than a first threshold value A1. When the sleep efficiency is lower than the first threshold value A1 and the sleep efficiency is equal to or higher than a second threshold value A2 (first threshold value A1>second threshold value A2), the determination circuit 62 determines that the user is rank 3. When the sleep efficiency is lower than the second threshold value A2, the determination circuit 62 determines that the user is rank 5. Determination of a rank may be made based on sleeping time as shown in FIG. 5B. FIGS. 6A-6C show data structures of alternative tables stored in the determination circuit 62. FIG. 6A shows a table used when the biological information includes sleepiness information, FIG. 6B shows a table used when the biological information includes emotion information, and FIG. 6C shows a table used when the biological information includes physical condition information. Reference is made back to FIG. 1.

Subsequently, the determination circuit 62 refers to the table stored in the storage circuit 50 and acquires information on vehicles of the rank corresponding to the rank of the user as determined (also called “user's rank”). When the user is rank “1”, for example, the determination circuit 62 acquires “all vehicles”. When the user is rank “3”, the determination circuit 62 acquires “automatically driven vehicles and support cars”. When the user is rank “5”, the determination circuit 62 acquires “not available”. Thus, the determination circuit 62 associates the rank with the user such that the better the biological information, the higher the rank. Further, the determination circuit 62 associates the user with one of the plurality of ranks based on the biological information and determines to lend a vehicle based on the associated rank.

The control circuit 52 generates a signal including the information on vehicles thus acquired (hereinafter, “rank signal”) and outputs the rank signal to the communication circuit 54. The communication circuit 54 transmits the rank signal to the second electronic device 30b via the network 20. In other words, when the biological information meets the first condition, the determination circuit 62 causes the communication circuit 54 to output information to offer to lend a vehicle of the first rank. Further, when the biological information meets the second condition different from the first condition, the determination circuit 62 causes the communication circuit 54 to output information to offer to lend a vehicle of the second rank. In the case the first rank is rank “1”, the second rank is rank “3” or “5”. In the case the first rank is rank “3”, the second rank is rank “5”. The first condition or the second condition is a condition to determine the first rank or the second rank. Further, the “rank signal” transmitted from the communication circuit 54 can be said to information to offer to lend a vehicle of the first rank or information to offer to lend a vehicle of the second rank. The storage circuit 50 can store the rank of each user and the biological information on each user.

The second electronic device 30b receives the rank signal. When the rank signal indicates “all vehicles”, the second electronic device 30b displays a screen shown in FIG. 4C. This represents a screen in the case of rank 1. A “Proceed” button and a “Pause” button are shown along with a message “You can select from all vehicles”. When the user selects the “Proceed” button, the second electronic device 30b transmits a signal indicating that the system should proceed with the process (hereinafter, “proceed signal”) to the vehicle lending control device 10. The control circuit 52 of the vehicle lending control device 10 receiving the proceed signal selects a vehicle from all vehicles, for which a publicly known technology may be used, and a description thereof is omitted. When the user selects the “Pause” button, the process is terminated.

When the rank signal indicates “automatically driven vehicles and support cars”, the second electronic device 30b displays a screen shown in FIG. 4D. This represents a screen in the case of rank 3. A “Proceed” button and a “Pause” button are shown along with a message “You can select from automatically driven vehicles and support cars”. When the user selects the “Proceed” button, the second electronic device 30b transmits a proceed signal to the vehicle lending control device 10. The control circuit 52 of the vehicle lending control device 10 receiving the proceed signal selects a vehicle from automatically driven vehicles and support cars, for which a publicly known technology may be used, and a description thereof is omitted. When the user selects the “Pause” button, the process is terminated.

When the rank signal indicates “not available”, the second electronic device 30b displays a screen shown in FIG. 4E. This represents a screen in the case of rank 5. An “End” button is shown along with a message “No vehicles are available for selection”. When the user selects the “End” button, the process is terminated.

The features are implemented in hardware such as a central processing unit (CPU), a memory, or other large scale integration (LSI) of an arbitrary computer and in software such as a program loaded into a memory. The figure depicts functional blocks implemented by the cooperation of these elements. Therefore, it will be understood by those skilled in the art that the functional blocks may be implemented in a variety of manners by hardware only or by a combination of hardware and software.

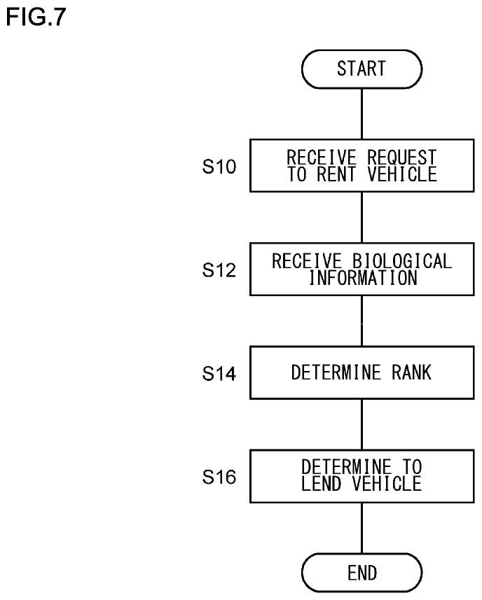

A description will be given of the operation of the vehicle lending control device 10 having the above-described configuration. FIG. 7 is a flowchart showing a sequence of steps for determination by the vehicle lending control device 10. The control circuit 52 receives a request to rent a vehicle from the second electronic device 30b (step S10). When the request to rent a vehicle is received, the reception circuit 60 receives biological information from the second electronic device 30b (step S12). The determination circuit 62 determines a rank of the user based on the biological information (step S14) and determines to lend a vehicle based on the rank of the user (step S16).

A description will now be given of a variation. It has been described above that the rank of a user is determined only by using biological information. In the variation, information other than biological information is also used. When the user drives a vehicle other than the plurality of vehicles available for rent, the user causes the second electronic device 30b to store driving information. The driving information may be behavior information described above. It is assumed here that the better the driving behavior of the user, the higher the value of the driving information. The driving information may be directly stored in the second electronic device 30b or may be stored in a device mounted in a further vehicle and then transferred to the second electronic device 30b. The second electronic device 30b includes the biological information and the driving information in the information signal and transmits the information signal to the vehicle lending control device 10.

When the communication circuit 54 of the vehicle lending control device 10 receives the information signal from the second electronic device 30b, the communication circuit 54 outputs the information signal to the control circuit 52. The reception circuit 60 receives the information signal from the communication circuit 54. This is equivalent to receiving information related to the driving information produced in the case the user drives a vehicle other than the plurality of vehicles available for rent.

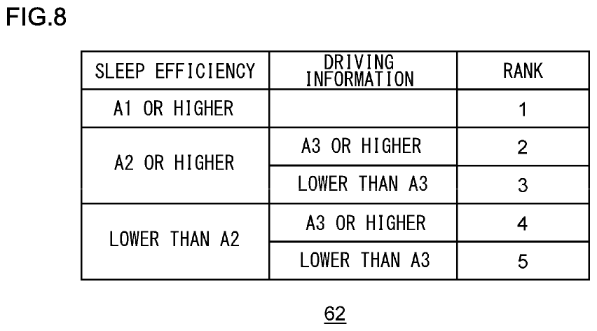

The determination circuit 62 determines a rank based on the sleep efficiency in the biological information and the driving information. FIG. 8 shows a data structure of a table stored in the determination circuit 62. The determination circuit 62 determines the user is rank 1 when the sleep efficiency is equal to or higher than a first threshold value A1. When the sleep efficiency is lower than the first threshold value A1 and the sleep efficiency is equal to or higher than a second threshold value A2 (first threshold value A1>second threshold value A2), the determination circuit 62 determines that the user is rank 2 provided that the driving information is of a value equal to or higher than a third threshold value A3. Meanwhile, when the driving information is of a value lower than the third threshold value A3 in that case, the determination circuit 62 determines that the user is rank 3. When the sleep efficiency is lower than the second threshold value A2, the determination circuit 62 determines that the user is rank 4 if the driving information is of a value equal to or higher than the third threshold value A3 and determines that the user is rank 5 if the driving information is of a value lower than third threshold value A3. In this way, the user is grouped into one of rank 1 through rank 5. In other words, the determination circuit 62 causes the communication circuit 54 to output, based on driving information produced in the case the user uses a vehicle different from the vehicle corresponding to the information to offer to lend a vehicle, information to offer to lend a vehicle of the first rank or information to offer to lend a vehicle of the second rank. In this case, the storage circuit 50 also groups the plurality of vehicles available for rent in association with rank 1 through rank 5 of the user.

According to this embodiment, the user is associated with one of a plurality of ranks based on biological information related to the condition of life of the user outside the vehicle, and a determination to lend a vehicle is made based on the associated rank. Accordingly, it is possible to impose a restriction on the use of a vehicle by a user regardless of whether the user has used a vehicle in the past. Further, a restriction on the use of a vehicle by a user is imposed regardless of whether the user has used a vehicle in the past. Accordingly, it is possible to impose a restriction on the use of a vehicle by a first-time user. Further, it is ensured that the higher the rank, the larger the number of vehicles included in the rank, and that the better the biological information, the higher the rank the user is associated with. Accordingly, it is ensured that the better the biological information on the user, the larger the number of vehicles that can be selected. Further, it is ensured that the lower the rank, the smaller the number of vehicles included in the rank, and that the poorer the biological information, the lower the rank the user is associated with. Accordingly, it is ensured that the poorer the biological information on the user, the more severely the use of a vehicle is restricted.

Further, the first electronic device and the second electronic device are used. Accordingly, an offer to lend a vehicle and the biological information can be transmitted. Further, the biological information on the user enters the vehicle lending control device from the first electronic device via the second electronic device. Accordingly, the biological information can be delivered to the vehicle lending control device. Further, a vital sensor adapted to acquire biological information on a user is provided. Accordingly, biological information can be acquired. Further, the information to offer to lend a vehicle of the first rank and the information to offer to lend a vehicle of the second rank are output to the second electronic device. Accordingly, the information can be presented to the user. Further, the information indicating application to rent a vehicle is received before the biological information. Accordingly, the process can be performed efficiently. Further, biological information may be one of information on sleep efficiency, sleepiness information, emotion information, and physical condition information. Accordingly the flexibility of the configuration can be improved. Further, driving information produced when the user drives a vehicle other than the plurality of vehicles available for rent is also used. Accordingly, the precision of determination of a rank can be improved. Further, at least the first rank, the second rank, and the biological information on the given user are stored. Accordingly, the process can be performed efficiently. Further, the information to offer to lend a vehicle of the first rank or the information to offer to lend a vehicle of the second rank is output in accordance with the first condition or the second condition. Accordingly, the information to offer to lend a vehicle can be transmitted to the user.

One embodiment of the present disclosure is summarized below. The vehicle lending control device according to an embodiment of the present disclosure is a vehicle lending control device including at least a communication circuit adapted to communicate with an electronic device, wherein a first rank related to a vehicle and a second rank related to a vehicle and lower than the first rank are provided. When the communication circuit receives, from the electronic device, information indicating application to rent a vehicle by a given user and biological information on the given user, and when the biological information on the given user meets a first condition, the communication circuit outputs information to offer to lend a vehicle of the first rank. When the communication circuit receives, from the electronic device, information indicating application to rent a vehicle by a given user and biological information on the given user, and when the biological information on the given user meets a second condition different from the first condition, the communication circuit outputs information to offer to lend a vehicle of the second rank.

According to this embodiment, the user is associated with one of a plurality of ranks based on biological information, and a determination to lend a vehicle is made based on the associated rank. Accordingly, it is possible to impose a restriction on the use of a vehicle by a user regardless of whether the user has used a vehicle in the past.

In the vehicle lending control device, the electronic device may include at least a first electronic device and a second electronic device, the first electronic device may acquire biological information on the user, and the second electronic device may include a user operation interface. The user operation interface of the second electronic device may be adapted to receive an application to rent a vehicle from the user. In this case, the first electronic device and the second electronic device are used. Accordingly, an application to rent a vehicle and biological information can be transmitted.

In the vehicle lending control device, the biological information on the user acquired by the first electronic device enters the communication circuit of the vehicle lending control device via the second electronic device. In this case, the biological information on the user enters the vehicle lending control device from the first electronic device via the second electronic device. Accordingly, the biological information can be delivered to the vehicle lending control device.

In the vehicle lending control device, the first electronic device may include a sensor adapted to acquire biological information on a user. In this case, a sensor adapted to acquire biological information on a user is provided. Accordingly, biological information can be acquired.

In the vehicle lending control device, the information to offer to lend a vehicle of the first rank and the information to offer to lend a vehicle of the second rank may be output by the communication circuit to the second electronic device. In this case, the information to offer to lend a vehicle of the first rank and the information to offer to lend a vehicle of the second rank are output to the second electronic device. Accordingly, the information can be presented to the user.

In the vehicle lending control device, the communication circuit receives the information indicating application to rent a vehicle by the given user before the biological information on the given user. In this case, the information indicating application to rent a vehicle is received before the biological information. Accordingly, the process can be performed efficiently.

In the vehicle lending control device, the biological information may be one of (1) information on sleeping time indicating actual sleeping time, (2) information on sleep efficiency indicating actual depth of sleep (REM/non-REM), (3) information indicating a proportion of a period of time in which a driver opens and closes the eyes during driving, (4) information indicating a proportion of a period of time in which the driver yawns (opens a mouth wider than a certain width), (5) sleepiness information acquired from the amount of heat dissipated from the body during driving, (6) emotion information acquired from a facial expression during driving and indicating a proportion of a period of time in which the driver is feeling bad (angry/sad), (7) emotion information acquired from the voice during driving and indicating a proportion of a period of time in which the driver is feeling bad (angry/sad), and (8) physical condition information acquired from the tilt of the head during driving and indicating a proportion of a period of time in which the physical condition becomes poorer. In this case, biological information may be one of information on sleep efficiency, sleepiness information, emotion information, and physical condition information. Accordingly the flexibility of the configuration can be improved.

In the vehicle lending control device, the communication circuit outputs, based on driving information produced in the case the user uses a vehicle different from the vehicle corresponding to the information to offer to lend a vehicle, the information to offer to lend a vehicle of the first rank or the information to offer to lend a vehicle of the second rank. In this case, driving information produced when the user drives a vehicle other than the plurality of vehicles available for rent is also used. Accordingly, the precision of determination of a rank can be improved.

The vehicle lending control device may further include: a storage circuit. The storage circuit may be adapted to store at least the first rank, the second rank, and the biological information on the given user. In this case, at least the first rank, the second rank, and the biological information on the given user are stored. Accordingly, the process can be performed efficiently.

The vehicle lending control device may further include: a control circuit. When the communication circuit receives, from the electronic device, the information indicating application to rent a vehicle by the given user and the biological information on the given user, and when the biological information on the given user meets the first condition, the control circuit may cause the communication circuit to output the information to offer to lend a vehicle of the first rank, and, when the communication circuit receives, from the electronic device, the information indicating application to rent a vehicle by the given user and the biological information on the given user, and when the biological information on the given user meets the second condition, the control circuit may cause the communication circuit to output the information to offer to lend a vehicle of the second rank. In this case, the information to offer to lend a vehicle of the first rank or the information to offer to lend a vehicle of the second rank is output in accordance with the first condition or the second condition. Accordingly, the information to offer to lend a vehicle can be transmitted to the user.

Another embodiment of the present disclosure relates to a vehicle lending control method. The method is a vehicle lending control method that can be used in a vehicle lending control device including at least a communication circuit adapted to communicate with an electronic device, wherein a first rank related to a vehicle and a second rank related to a vehicle and lower than the first rank are provided. When the communication circuit receives, from the electronic device, information indicating application to rent a vehicle by a given user and biological information on the given user, and when the biological information on the given user meets a first condition, the communication circuit outputs information to offer to lend a vehicle of the first rank, and, when the communication circuit receives, from the electronic device, information indicating application to rent a vehicle by a given user and biological information on the given user, and when the biological information on the given user meets a second condition different from the first condition, the communication circuit outputs information to offer to lend a vehicle of the second rank.

According to this embodiment, the user is associated with one of a plurality of ranks based on biological information, and a determination to lend a vehicle is made based on the associated rank. Accordingly, it is possible to impose a restriction on the use of a vehicle by a user regardless of whether the user has used a vehicle in the past.

In the vehicle lending control method, the electronic device may include at least a first electronic device and a second electronic device, the first electronic device may acquire biological information on the user, and the second electronic device may include a user operation interface. The user operation interface of the second electronic device may be adapted to receive an application to rent a vehicle from the user. In this case, the first electronic device and the second electronic device are used. Accordingly, an offer to lend a vehicle and the biological information can be transmitted.

In the vehicle lending control method, the biological information on the user acquired by the first electronic device enters the communication circuit of the vehicle lending control device via the second electronic device. In this case, the biological information on the user enters the vehicle lending control device from the first electronic device via the second electronic device. Accordingly, the biological information can be delivered to the vehicle lending control device.

In the vehicle lending control method, the first electronic device may include a sensor adapted to acquire biological information on a user. In this case, a sensor adapted to acquire biological information on a user is provided. Accordingly, biological information can be acquired.

In the vehicle lending control method, the information to offer to lend a vehicle of the first rank and the information to offer to lend a vehicle of the second rank are output by the communication circuit to the second electronic device. In this case, the information to offer to lend a vehicle of the first rank and the information to offer to lend a vehicle of the second rank are output to the second electronic device. Accordingly, the information can be presented to the user.

In the vehicle lending control method, the communication circuit receives the information indicating application to rent a vehicle by the given user before the biological information on the given user. In this case, the information indicating application to rent a vehicle is received before the biological information. Accordingly, the process can be performed efficiently.

In the vehicle lending control method, the biological information may be one of (1) information on sleeping time indicating actual sleeping time, (2) information on sleep efficiency indicating actual depth of sleep (REM/non-REM), (3) information indicating a proportion of a period of time in which a driver opens and closes the eyes during driving, (4) information indicating a proportion of a period of time in which the driver yawns (opens a mouth wider than a certain width), (5) sleepiness information acquired from the amount of heat dissipated from the body during driving, (6) emotion information acquired from a facial expression during driving and indicating a proportion of a period of time in which the driver is feeling bad (angry/sad), (7) emotion information acquired from the voice during driving and indicating a proportion of a period of time in which the driver is feeling bad (angry/sad), and (8) physical condition information acquired from the tilt of the head during driving and indicating a proportion of a period of time in which the physical condition becomes poorer. In this case, biological information may be one of information on sleep efficiency, sleepiness information, emotion information, and physical condition information. Accordingly the flexibility of the configuration can be improved.

In the vehicle lending control method, the communication circuit may output, based on driving information produced in the case the user uses a vehicle different from the vehicle corresponding to the information to offer to lend a vehicle, the information to offer to lend a vehicle of the first rank or the information to offer to lend a vehicle of the second rank. In this case, driving information produced when the user drives a vehicle other than the plurality of vehicles available for rent is also used. Accordingly, the precision of determination of a rank can be improved.

The vehicle lending control method may further include: a storage circuit. The storage circuit may be adapted to store at least the first rank, the second rank, and the biological information on the given user. In this case, at least the first rank, the second rank, and the biological information on the given user are stored. Accordingly, the process can be performed efficiently.

The vehicle lending control method may further include: a control circuit. When the communication circuit receives, from the electronic device, the information indicating application to rent a vehicle by the given user and the biological information on the given user, and when the biological information on the given user meets the first condition, the control circuit causes the communication circuit to output the information to offer to lend a vehicle of the first rank, and, when the communication circuit receives, from the electronic device, the information indicating application to rent a vehicle by the given user and the biological information on the given user, and when the biological information on the given user meets the second condition, the control circuit causes the communication circuit to output the information to offer to lend a vehicle of the second rank. In this case, the information to offer to lend a vehicle of the first rank or the information to offer to lend a vehicle of the second rank is output in accordance with the first condition or the second condition. Accordingly, the information to offer to lend a vehicle can be transmitted to the user.

Described above is an explanation based on an exemplary embodiment. The embodiment is intended to be illustrative only and it will be understood by those skilled in the art that various modifications to constituting elements and processes could be developed and that such modifications are also within the scope of the present disclosure.

In the variation, the rank of a user is determined based on the biological information and the driving information. Alternatively, however, the rank of a user may be determined without using the biological information and by using the driving information. According to this variation, the behavior of the user driving a vehicle other than the plurality of vehicles available for rent can be reflected in the ranking.

While various embodiments have been described herein above, it is to be appreciated that various changes in form and detail may be made without departing from the spirit and scope of the invention(s) presently or hereafter claimed.

### CROSS-REFERENCE TO RELATED APPLICATION

This application is based upon and claims the benefit of priority from the prior Japanese Patent Application No. 2018-050148, filed on Mar. 16, 2018, the entire contents of which are incorporated herein by reference.

What is claimed is:

1. A vehicle lending control device comprising at least a communication circuit adapted to communicate with an electronic device, wherein
a first rank related to a vehicle and
a second rank related to a vehicle and lower than the first rank are provided,
when the communication circuit receives, from the electronic device, information indicating application to rent a vehicle by a given user and biological information on the given user, and when the biological information on the given user meets a first condition, the communication circuit outputs information to offer to lend a vehicle of the first rank, and
when the communication circuit receives, from the electronic device, information indicating application to rent a vehicle by a given user and biological information on the given user, and when the biological information on the given user meets a second condition different from the first condition, the communication circuit outputs information to offer to lend a vehicle of the second rank.

2. The vehicle lending control device according to claim 1, wherein
the electronic device includes at least a first electronic device and a second electronic device,
the first electronic device acquires biological information on the user,
the second electronic device includes a user operation interface, and
the user operation interface of the second electronic device is adapted to receive an application to rent a vehicle from the user.

3. The vehicle lending control device according to claim 2, wherein
the biological information on the user acquired by the first electronic device enters the communication circuit of the vehicle lending control device via the second electronic device.

4. The vehicle lending control device according to claim 2, wherein
the first electronic device includes a sensor adapted to acquire biological information on a user.

5. The vehicle lending control device according to claim 2, wherein
the information to offer to lend a vehicle of the first rank and the information to offer to lend a vehicle of the second rank are output by the communication circuit to the second electronic device.

6. The vehicle lending control device according to claim 1, wherein
the communication circuit receives the information indicating application to rent a vehicle by the given user before the biological information on the given user.

7. The vehicle lending control device according to claim 1, wherein
the biological information is one of
(1) information on sleeping time indicating actual sleeping time,
(2) information on sleep efficiency indicating actual depth of sleep (REM/non-REM),
(3) information indicating a proportion of a period of time in which a driver opens and closes the eyes during driving,
(4) information indicating a proportion of a period of time in which the driver yawns (opens a mouth wider than a certain width),
(5) sleepiness information acquired from the amount of heat dissipated from the body during driving,
(6) emotion information acquired from a facial expression during driving and indicating a proportion of a period of time in which the driver is feeling bad (angry/sad),
(7) emotion information acquired from the voice during driving and indicating a proportion of a period of time in which the driver is feeling bad (angry/sad), and
(8) physical condition information acquired from the tilt of the head during driving and indicating a proportion of a period of time in which the physical condition becomes poorer.

8. The vehicle lending control device according to claim 1, wherein
the communication circuit outputs, based on driving information produced in the case the user uses a vehicle different from the vehicle corresponding to the information to offer to lend a vehicle, the information to offer to lend a vehicle of the first rank or the information to offer to lend a vehicle of the second rank.

9. The vehicle lending control device according to claim 1, further comprising:
a storage circuit, wherein
the storage circuit is adapted to store at least the first rank, the second rank, and the biological information on the given user.

10. The vehicle lending control device according to claim 1, further comprising:
a control circuit, wherein
when the communication circuit receives, from the electronic device, the information indicating application to rent a vehicle by the given user and the biological information on the given user, and when the biological information on the given user meets the first condition, the control circuit causes the communication circuit to output the information to offer to lend a vehicle of the first rank, and
when the communication circuit receives, from the electronic device, the information indicating application to rent a vehicle by the given user and the biological information on the given user, and when the biological information on the given user meets the second condition, the control circuit causes the communication circuit to output the information to offer to lend a vehicle of the second rank.

11. A vehicle lending control method that can be used in a vehicle lending control device including at least a communication circuit adapted to communicate with an electronic device, wherein
a first rank related to a vehicle and
a second rank related to a vehicle and lower than the first rank are provided,
when the communication circuit receives, from the electronic device, information indicating application to rent a vehicle by a given user and biological information on the given user, and when the biological information on the given user meets a first condition, the communication circuit outputs information to offer to lend a vehicle of the first rank, and
when the communication circuit receives, from the electronic device, information indicating application to rent a vehicle by a given user and biological information on the given user, and when the biological information on the given user meets a second condition different from the first condition, the communication circuit outputs information to offer to lend a vehicle of the second rank.

12. The vehicle lending control method according to claim 11, wherein
the electronic device includes at least a first electronic device and a second electronic device,
the first electronic device acquires biological information on the user,
the second electronic device includes a user operation interface, and
the user operation interface of the second electronic device is adapted to receive an application to rent a vehicle from the user.

13. The vehicle lending control method according to claim 12, wherein
the biological information on the user acquired by the first electronic device enters the communication circuit of the vehicle lending control device via the second electronic device.

14. The vehicle lending control method according to claim 12, wherein
the first electronic device includes a sensor adapted to acquire biological information on a user.

15. The vehicle lending control method according to claim 12, wherein
the information to offer to lend a vehicle of the first rank and the information to offer to lend a vehicle of the second rank are output by the communication circuit to the second electronic device.

16. The vehicle lending control method according to claim 11, wherein
the communication circuit receives the information indicating application to rent a vehicle by the given user before the biological information on the given user.

17. The vehicle lending control method according to claim 11, wherein
the biological information is one of
(1) information on sleeping time indicating actual sleeping time,
(2) information on sleep efficiency indicating actual depth of sleep (REM/non-REM),
(3) information indicating a proportion of a period of time in which a driver opens and closes the eyes during driving,
(4) information indicating a proportion of a period of time in which the driver yawns (opens a mouth wider than a certain width),
(5) sleepiness information acquired from the amount of heat dissipated from the body during driving,
(6) emotion information acquired from a facial expression during driving and indicating a proportion of a period of time in which the driver is feeling bad (angry/sad),
(7) emotion information acquired from the voice during driving and indicating a proportion of a period of time in which the driver is feeling bad (angry/sad), and
(8) physical condition information acquired from the tilt of the head during driving and indicating a proportion of a period of time in which the physical condition becomes poorer.

18. The vehicle lending control method according to claim 11, wherein
the communication circuit outputs, based on driving information produced in the case the user uses a vehicle different from the vehicle corresponding to the information to offer to lend a vehicle, the information to offer to lend a vehicle of the first rank or the information to offer to lend a vehicle of the second rank.

19. The vehicle lending control method according to claim 11, further comprising:
a storage circuit, wherein
the storage circuit is adapted to store at least the first rank, the second rank, and the biological information on the given user.

20. The vehicle lending control method according to claim 11, further comprising:
a control circuit, wherein
when the communication circuit receives, from the electronic device, the information indicating application to rent a vehicle by the given user and the biological information on the given user, and when the biological information on the given user meets the first condition, the control circuit causes the communication circuit to output the information to offer to lend a vehicle of the first rank, and
when the communication circuit receives, from the electronic device, the information indicating application to rent a vehicle by the given user and the biological information on the given user, and when the biological information on the given user meets the second condition, the control circuit causes the communication circuit to output the information to offer to lend a vehicle of the second rank.
